(19) United States
(12) Patent Application Publication
(10) Pub. No.: US 2002/0151491 A1
(43) Pub. Date: Oct. 17, 2002

(54) Composition and method for treating the over-production of mucin in diseases such as otitis media using an inhibitor of MUC5AC

(76) Inventors: Jian-Dong Li, Glendale, CA (US); David Lim, Pasadena, CA (US); Haidong Xu, Glendale, CA (US); Beinan Wang, Glendale, CA (US); Tsuyoshi Shuto, Kumamoto (JP); Carol Basbaum, San Francisco, CA (US); Young S. Kim, Hillsborough, CA (US)

Correspondence Address: KNOBBE MARTENS OLSON & BEAR LLP, 2040 MAIN STREET, FOURTEENTH FLOOR, IRVINE, CA, 91614, US

(21) Appl. No.: 09/997,551

(22) Filed: Nov. 27, 2001

Related U.S. Application Data
(60) Provisional application No. 60/253,494, filed on Nov. 28, 2000.

(51) Int. Cl. A61K 48/00; A61K 38/17; A61K 31/519; A61K 31/506

(52) U.S. Cl. 514/12; 514/44; 514/256; 514/259.1

(57) ABSTRACT

Disclosed herein is a method for the identification of a treatment for overproduction of mucin during otitis media (OM) and chronic obstructive pulmonary disease (COPD). The method uses a MUC5AC plasmid to identify novel cytoplasmic proteins of Nontypeable Haemophilus influenzae, a common mediator of OM and COPD, which up-regulate human MUC5AC mucin transcription via a positive p38 MAP kinase pathway and a negative PI 3-Kinase-Akt pathway. These proteins can be used to identify or design inhibitors of the p38 MAP kinase pathway and activators of the PI 3-kinase Akt pathway.

### BACKGROUND OF THE INVENTION

1. Field of the Invention

The present invention provides for methods of identifying compounds for treating medical conditions related to the inappropriate overproduction of mucin in the middle ear and respiratory system, as well as compounds and methods for treating such conditions. More specifically, the present invention identifies methods of treating mucin overproduction with P38 MAP kinase inhibitors or PI 3 kinase activators.

2. Description of the Related Art

The overproduction of mucin is associated with diseases such as Otitis media (OM), the most common childhood infection and also the leading cause of conductive hearing loss in children, and chronic obstructive pulmonary disease (COPD), a lower respiratory tract infection and the fourth leading cause of death in the United States. While it has been shown that overproduction of mucin, the major protein of mucus in the middle ear, plays an important role in the development of conductive hearing loss, little is known about the causes of and molecular mechanisms underlying mucin overproduction. Moreover, inappropriate antibiotic treatment of OM contributes to the worldwide emergence of multidrug-resistant strains of bacterial pathogens. Thus, due to the prevalence, long-term sequelae and the cost to our society, there is an urgent need for the development of novel therapeutic strategies.

Nontypeable Haemophilus influenzae (NTHi) is an important human pathogen in both children and adults. In children, it causes otitis media (OM), the most common childhood infection and the leading cause of conductive hearing loss in the United States. In adults, it causes lower respiratory tract infections in the setting of chronic obstructive pulmonary disease (COPD). The molecular mechanisms underlying the pathogenesis of NTHi-induced infections remain undefined.

Although significant progress has been made toward identifying the virulence factors of NTHi, the molecular pathogenesis of NTHi infections is still largely unknown. Interestingly, there is evidence that up-regulation of mucin production induced by bacteria could play an important role. Mucins are high-molecular weight glycoproteins that constitute the major component of mucus secretions in the middle ear, trachea, digestive and reproductive tracts. They protect and lubricate the epithelial surface and trap particles, including bacteria and viruses, for mucociliary clearance. In COME and COPD, excessive production of mucin occurs, overwhelming the normal mucociliary clearance mechanisms. As mucus levels increase, they contribute significantly to airway obstruction in COPD and conductive hearing loss in COME. In addition to the obstructive outcome, mucin has been reported to bind to almost all known bacterial pathogens. The combination of defective mucociliary clearance and mucin-bacteria interaction could greatly increase the ability of bacteria to persist in a host. To date, 13 mucin genes have been cloned and one, MUC5AC, has been shown to be highly expressed in airway and middle ear epithelial cells. Furthermore, recent studies have demonstrated that the expression level of MUC5AC mRNA in the middle ear is higher in patients with COME than in normal individuals. Taken together, these studies strongly suggest that up-regulation of the MUC5AC mucin gene plays an important role in the pathogenesis of NTHi infections.

Although little is known about how NTHi up-regulates MUC5AC mucin transcription, previous studies have shown that bacteria can activate transcription of host defense genes via activation of specific signal transduction cascades. Among the commonly known signaling events, the mitogen-activated protein kinase (MAP kinase) pathways are thought to be most important in transmitting extracellular signals from the cell surface to the nucleus. p38, a major MAP kinase superfamily member, has been shown to be involved in NTHi-induced inflammatory responses. In addition to p38 MAP kinase, phosphoinositide 3-kinase (PI 3-kinase) represents another major signaling transducer involved in the regulation of cell proliferation, survival, metabolism, cytoskeleton reorganization and membrane trafficking as well as bacterial pathogenesis. However, the role of both p38 MAP kinase and PI-3 kinase in mucin up-regulation has not yet been explored.

### SUMMARY OF THE INVENTION

The present invention now recognizes for the first time an important role of p38 MAP kinase, a key signaling molecule involved in cellular stress responses, in nontypeable Haemophilus influenzae (NTHi)-induced mucin MUC5AC overproduction.

The information provided by the pathway is used in methods for identifying compounds for inhibiting mucin overproduction in middle ear. In these methods, a MUC5AC promoter luciferase construct was transfected into human epithelial cells including middle ear epithelial cells. The stably transfected cell line was used for identifying the pathway of NTHi-induced mucin production. In this way, the p38 MAP kinase was identified as being involved in the mucin production.

Thus, one embodiment of the invention provides for methods for inhibiting the overproduction of mucin by cells, such as middle ear cells, by applying an effective amount of SB203580 or related compounds, a potent inhibitor of p38 MAP kinase, to the epithelial cells in middle ear. To date, there has been no report on the role of p38 MAP kinase in mucin overproduction in middle ear. Thus, any compound developed based on the pyridinyl imidazole structure of SB203580 can be used for the inhibition of mucus overproduction to prevent conductive hearing loss and recurrent infection in otitis media.

A further embodiment is a method for the treatment of overproduction of mucin in a mammal, by administering an inhibitor of p38 MAP kinase to the mammal in an amount sufficient to reduce mucin production. In one embodiment, the overproduction of mucin is caused by an otitis media (OM) infection or chronic obstructive pulmonary disease (COPD), particularly by nontypeable Haemophilus influenzae (NTHi).

In one embodiment, the inhibitor of p38 MAP kinase is a chemical inhibitor selected from the group consisting of: pyridimylimidzol SB203580, SB202190, SB220025, SC68376, SKF-86002, a dominant-negative mutant of p38α, and a dominant-negative mutant of p38β. The inhibitor may be an antisense oligonucleotide, a vector which expresses a protein or polypeptide which inhibits p38 MAP kinase, a transcription factor which binds to the p38 promoter, or a protein which binds to the p38 protein. In one embodiment, the method of administration is selected from the group consisting of: inhalation, ear drops, transtympanically, intramuscularly, intravenously, and by mouth.

A further embodiment is a method for the identification of regulators of mucin production, by providing a reporter vector containing the MUC5AC or p38 MAP kinase promoter, contacting the reporter vector with a potential regulator; and identifying the up-or down-regulation of the reporter gene.

In one embodiment, the potential regulator is selected from the group consisting of: a polypeptide, an polynucleotide, and a small molecule. In a further embodiment, the potential regulator is a mixture of proteins from a cell or an antisense polynucleotide or a library of small molecules.

A further embodiment is a method for the treatment of overproduction of mucin in a mammal, by administering an activator of PI-3 kinase to the mammal in an amount sufficient to reduce mucin production. In one embodiment, the overproduction of mucin is caused by a disease selected from the group consisting of: Otitis media, chronic obstructive pulmonary disease, asthma, and cystic fibrosis. In a further embodiment the overproduction of mucin is caused by otitis media (OM) infection or chronic obstructive pulmonary disease (COPD), particularly caused by nontypeable Haemophilus influenzae (NTHi).

In one embodiment, the activator of PI-3 kinase is a protein selected from the group consisting of: a dominant negative mutant of PI-3 kinase, a constitutively active form of p110 (p110-CAAX), wildtype Akt, an antisense oligonucleotide, and a vector which expresses a protein or polypeptide which activates PI-3 kinase.

In a further embodiment, the method of administration is selected from the group consisting of: inhalation, ear drops transtympanically, intramuscularly, intravenously, and by mouth.

### BRIEF DESCRIPTION OF THE DRAWINGS

FIG. 1 shows the up-regulation of MUC5AC mucin gene transcription by NTHi. (A) shows up-regulation of MUC5AC expression at the mRNA level. HeLa (human cervix epithelial) cells were treated with or without NTHi sonicated bacteria in duplicate for 5 h. RT-PCR was then performed to measure the changes in steady-state mRNA levels. Cyclophilin served as a control for the amount of RNA used in each reaction. Similar results were also observed in HM3 (human colon epithelial) cells. Data represent four independent experiments. (B) shows up-regulation of MUC5AC transcription in human epithelial cells. A 3.7-kb DNA fragment of the 5′-flanking region of the human MUC5AC mucin gene cloned into a luciferase reporter vector (pMWC5AC3.7luc) was transfected into HeLa, HM3 and A549 (human airway epithelial) cells. Luciferase activity was then assessed in NTHi sonicated bacteria-treated and nontreated cells. Induction by NTHi was detected in all cell lines. (C) shows that all clinically isolated NTHi strains tested were capable of inducing MUC5AC transcription. HM3 cells stably transfected with pMUC5AC3.7luc were exposed to sonicated bacteria from various NTHi strains as indicated for 4h. Luciferase activity was then assessed in NTHi-treated and untreated cells. All transfections and luciferase assays were carried out in triplicate. Values represent means ±SD (n=3).

FIG. 2. Shows non-LOS molecules which were released from lysed NTHi by sonication are responsible for the potent MUC5AC-inducing activity. (A) shows the effects of various NTHi fractions on MUC5AC induction. HM3 cells stably transfected with pMUC5AC3.7luc were exposed to whole bacteria and various fractions from NTHi as indicated for 4 h. Luciferase activity was then assessed in NTHi-treated and untreated cells. WB, whole intact NTHi bacteria in PBS; SB, sonicated NTHi bacteria in PBS; SCF, soluble cytoplasmic fraction of sonicated bacteria after centrifugation at 10,000×g, 10 min; P, pellet of sonicated bacteria after centrifugation. (B) shows that NTHi LOS did not induce MUC5AC transcription. HM3 cells stably transfected with pMUC5AC3.7luc were treated with various concentrations of NTHi LOS as indicated for 4 h before being lysed for luciferase assay. (C) shows that polymyxin B treatment did not attenuate up-regulation of MUC5AC induced by NTHi soluble cytoplasmic components. NTHi soluble cytoplasmic fractions (SCF) were pretreated with various concentrations of polymyxin B for 10 min before being added to HM3 cells stably transected with pMUC5AC3.7luc. (D) shows that polymyxin B significantly reduced MUCSAC induction by LPS from Salmonella typhimurium. LPS was pretreated with various concentrations of polymyxin B for 10 min at 4° C. and was then added to HM3 cells stably transfected with pMUC5AC3.7luc for 4 h before being lysed for luciferase assay. All luciferase assays were carried out in triplicate. Values represent means ±SD (n=3).

FIG. 3 shows that cytoplasmic components of NTHi play a major role in MUC5AC induction. (A) shows that the MUC5AC-inducing activity of the cytoplasmic components of NTHi is much more potent than that of NTHi envelope proteins. Envelope proteins were separated from the cytoplasmic components by ultracentrifugation of sonicated NTHi. The cytoplasmic and envelope fractions were then added to HM3 cells stably transfected with pMUC5AC3.7luc for 4 h before luciferase assay. (B) shows that a similar potent MUC5AC-inducing activity was also observed in the cytoplasmic components, which were prepared from the disrupted NTHi using French Pressure cell, an alternative approach to completely disrupt the bacterial cells. NTHi cells were disrupted using French Pressure cell at 1,000 Psi. The cytoplasmic components were separated from the envelope components by centrifugation at 10,000×g at 4° C. for 10 min followed by ultracentrifugation at 1,000,000×g at 4° C. for 1 h. After centrifugation, the pellet (envelope components) and the cytoplasmic components were added to HM3 cells stably transected with pMUC5AC3.7luc for 4 h before being lysed for luciferase assay. Whole Bacteria, NTHi whole bacterial cells; Cyto, cytoplasmic components; EP, envelope proteins. All luciferase assays were carried out in triplicate. Values represent mean ±SD (n=3).

FIG. 4 shows that proteins were the major NTHi soluble cytoplasmic components responsible for MUC5AC induction. (A) shows that treatment with DNase and RNase does not reduce NTHi-induced MUC5AC transcription. NTHi soluble cytoplasmic fractions (SCF) were pretreated with either DNase (34 μg/ml) or RNase (50 μg/ml) or buffer alone overnight, and were then added to HM3 cells stably transected with pMUC5AC3.7luc for 4 h before being lysed for luciferase assay. (B) shows that proteins are the major MUC5AC inducers in NTHi soluble cytoplasmic components. The soluble cytoplasmic components were boiled at 100° C. for 5 min (Heat) or incubated at 37° C. overnight in the presence or absence of protease inhibitor cocktail (PI) (1.3 mg/ml). For PE and PBS groups, aliquots of overnight-incubated samples without protease inhibitor were further treated with protease E (PE) (300 μg/ml) or PBS alone as a control for another 2 h at 37° C. before being lysed for luciferase assay. All luciferase assays were carried out in triplicate in HM3 cells stably transected with pMUC5AC3.7luc. Values represent means ±SD (n=3)

FIG. 5 shows that activation of p38 MAP kinase is required for NTHi-induced MUC5AC transcription. (A) shows that NTHi SCF induces p38 MAP kinase phosphorylation in HM3 cells. (B) shows that SB203580, a specific inhibitor for p38 MAP kinase, attenuated NTHi SCF-induced MUC5AC transcription in a dosedependent manner. HM3 cells stably transected with pMUC5AC3.7luc were pretreated with SB203580 for 1h and were then treated with NTHi SCF for 4h before being lysed for luciferase assay. (C) shows that overexpression of a dominant-negative mutant of either p38α or p38β inhibited NTHi-induced MUC5AC transcription as follows: A dominant-negative mutant of either p38α (p38α DN) or p38β(p38β DN) was transiently co-transected into HM3 cells with pMUC5AC3.7lu. After 42 h, the transfected cells were treated with or without NTHi SCF for 4 h. The cells were then lysed and assayed for luciferase activity. An empty vector served as a control. All transfections and luciferase assays were carried out in triplicate. Values represent means ±SD (n=3).

FIG. 6 shows that PI 3-kinase is negatively involved in NTHi-induced MUC5AC transcription. (A) LY294002, a specific inhibitor for PI 3-kinase, enhanced NTHi-induced MUC5AC transcription in a dose-dependent manner. HM3 cells stably transected with pMUC5AC3.7luc were pretreated with LY294002 for 2 h and were then treated with NTHi SCF for 4 h before being lysed for luciferase assay. (B) shows that wortmannin, another specific inhibitor for PI 3-kinase, also enhanced NTHi SCF-induced MUC5AC transcription in a dose-dependent manner. (C) shows that overexpression of a dominant-negative mutant of p110 (p110 KD), a catalytic subunit of PI-3 kinase, enhances, whereas overexpression of an activated, membrane-targeted form of p110 (p110-CAAX) attenuates, MUC5AC induction. (D) shows that overexpression of a dominant-negative mutant of p85α (p85α DN), a regulatory subunit of PI 3-kinase, enhances NTHi-induced MUC5AC transcription. All transient transfections were carried out in triplicate in HM3 cells and the transfected cells were then treated with NTHi SCF for 4 h. Values represent means ±SD (n=3).

FIG. 7 shows that PI 3-kinase dependent activation of Akt leads to down-regulation of NTHi-induced MUC5AC transcription via a negative cross-talk with p38 MAP kinase. (A) Upper Panel: shows that Akt is phosphorylated in response to the treatment of NTHi SCF. HM3 cells were treated with NTHi SCF, or PBS and lysed at various times for Western Blot analysis with antibodies against phospho-Akt and Akt. Lower Panel: Akt is phosphorylated in response to the treatment of various fractions of NTHi, including whole bacteria (WB), sonicated bacteria (SB), envelope proteins (EP) and SCF or PBS and lysed at 30 min for Western Blot analysis with antibodies against phospho-Akt and Akt. (B) shows that overexpression of a dominant-negative mutant of Akt (Akt KD) enhances, whereas overexpression of wild-type form of Akt (Akt WT) inhibits, MUC5AC induction. The transient transfections were carried out in BJM3 cells and the transfected cell were then treated with NTHi SCF for 4 h before being lysed for luciferase assay. (C) shows that NTHi SCF-induced Akt phosphorylation was abrogated by PI 3-Kinase inhibitor wortmannin (WM). HM3 cells were pretreated with wortmannin for 2 h and then incubated with NTHi SCF for 15 min, 30 min, respectively. Western Blot analysis was then carried out to measure the phosphorylation of Akt using antibodies against Akt and phosphorylated form of Akt. (D) shows that the PI 3-kinase inhibitor wortmannin greatly enhanced NTHi SCF-induced p38 MAP kinase phosphorylation in HM3 cells. (E) shows that overexpression of an activated form of p110 (p110-CAAX) attenuated NTHi SCF-induced phosphorylation of p38 MAP kinase in HM3 cells. (F) shows that wortmannin no longer enhanced NTHi SCF-induced MUC5AC transcription in HM3 cells stably transected with pMUC5AC3.7luc that have been already pretreated with SB203580. All luciferase assays were carried out in triplicate. Values represent means ±SD (n=3).

FIG. 8 is a schematic diagram showing the intracellular signaling pathways involved in NTHi-induced human mucin MUC5AC transcription. As indicated, the cytoplasmic proteins released from the lysed NTHi induce activation of p38 MAP kinase pathway and PI 3-kinase-Akt pathway. Activation of p38 is required for NTHi-induced MUC5AC transcription, whereas activation of PI 3-kinase-Akt pathway leads to down-regulation of NTHi-induced MUC5AC transcription via a negative cross-talk with p38 MAP kinase pathway. The overproduced mucin, in concert with defective mucociliary clearance, leads to airway mucus obstruction in chronic obstructive pulmonary diseases (COPD) and conductive hearing loss in chronic otitis media with effusion (COME).

### DETAILED DESCRIPTION OF THE PREFERRED EMBODIMENT

A method and pharmaceutical preparation for the treatment of the over production of mucin in OM and COPD is identified herein. The method and pharmaceutical preparation are based on the results herein which use NTHi as a prototypic microorganism to identify the molecular mechanisms of the overproduction of mucin. It is envisioned that other bacteria which activate mucin production may use the same pathways. Thus, the method and preparation herein may be used for the inhibition of mucin production in infections and may be useful for the treatment of mucin production during allergies.

Nontypeable Haemophilus influenzae (NTHi) is an important human pathogen that causes chronic otitis media with effusion (COME) in children and exacerbation of chronic obstructive pulmonary disease (COPD) in adults. Mucin overproduction, a hallmark of both diseases, has been shown to directly cause conductive hearing loss in COME and airway obstruction in COPD. The molecular mechanisms underlying mucin overproduction in NTHi infections still remain unclear. Therefore, using the method herein, the molecular mechanisms used by NTHi to up-regulate MUC5AC mucin transcription were identified to only occur after bacterial cell disruption. Maximal upregulation was induced by heat-stable bacterial cytoplasmic proteins, whereas NTHi surface membrane proteins induced only moderate MUC5AC transcription. These results demonstrate an important role for cytoplasmic molecules from lysed bacteria in the pathogenesis of NTHi infections, and may well explain why many patients still have persistent symptoms such as middle ear effusion in COME after intensive antibiotic treatment. Furthermore, the results indicate that activation of the p38 MAP kinase is required for NTHi-induced MUC5AC transcription, whereas activation of PI 3-kinase-Akt pathway leads to down-regulation of NTHi-induced MUC5AC transcription via a negative cross-talk with p38 MAP kinase pathway.

Thus, one embodiment of the invention provides methods for identifying compounds which inhibit mucin overproduction using a MUC5AC promoter-luciferase reporter construct. In this method, a MUC5AC promoter luciferase construct is transfected into human epithelial cells including middle ear epithelial cells. This stably transfected cell line is used for screening for any compounds that can inhibit NTHi-induced mucin overproduction. Other cell lines used in the method are the HMEEC-1 human middle ear epithelial cell line, and the HM3, a human mucin-expressing epithelial cell line for studying and identifying inhibitors of mucin production.

In a further embodiment, methods for identifying compounds which inhibit mucin production use a p38 MAP kinase promoter luciferase reporter construct or the equivalent. The p38 MAP kinase reporter construct is transfected into cell lines. This transfected cell line is used for screening for any compounds that can inhibit MUC5AC production by inhibiting p38 MAP kinase expression.

In a further embodiment, methods for identifying compounds which inhibit mucin production use a PI-3 kinase promoter luciferase reporter construct or the equivalent. The PI-3 kinase reporter construct is transfected into cell lines. This transfected cell line is used for screening for any compounds that can inhibit MUC5AC production by activating the PI-3 kinase expression.

In one embodiment, inhibitors of mucin production may be any polypeptide, polynucleotide, small molecule, pharmaceutical, or vector which inhibits mucin production by inhibiting the p38 MAP kinase pathway. In a further embodiment, inhibitors of mucin production may be any polypeptide, polynucleotide, small molecule, pharmaceutical, or vector which inhibits mucin production by activating the PI-3 kinase pathway. The inhibitors may act by binding to the promoters and affecting transcription, or the inhibitors may act by binding to the proteins themselves.

Inhibitors may be used to treat diseases which result in the overproduction of the MUC5AC mucin. Examples of such diseases include but are not limited to: infections of inner ear, sinuses, upper and lower respiratory tract, cystic fibrosis, asthma, and allergies. The most common example of diseases which result in the overproduction of mucin include but are not limited to COPD and COME. However, some previous results suggest that MUC5AC may be involved in the pathogenesis of asthma and airway hyperactivity.

The causative agents of these diseases may be any pathogens, including but not limited to: Haemophilus influenza, Streptococcus pneumonia, Moraxella catarrhalis, Mycoplasma pneumonia, and Chlamydia pneumonia. It is to be understood however, that although the most common causative agents of COPD and COME are currently H. influenza, S. pneumonia, and M. pneumonia, this may change from year to year and from region to region. One of skill in the art realizes that the most common causative agents may change by region or may change from year to year due to microbial evolution, to environmental changes, to the use or misuse of antibiotics, to the ability of microbes to mutate to infect a new host or to infect a new part of the body. Alternatively, microbes which were previously unable to activate mucin production may acquire this ability. Thus, the inhibitors herein may be used for diseases which are presently associated with mucin overproduction or which may evolve to be associated with mucin overproduction.

In a further embodiment, methods for identifying compounds which activate mucin MUC5AC production use a PI-3 kinase promoter luciferase reporter construct or the equivalent. The PI-3 kinase reporter construct is transfected into cell lines. This transfected cell line is used for screening for any compounds that can activate MUC5AC production by inhibiting the PI-3 kinase expression. The equivalent method is used to identify activators of the p38 pathway. Activators of the mucin MUC5AC production may also act in protein-protein interactions. These activators may be identified using methods known to one of skill in the art.

It is envisioned that activators of mucin production may be useful for any disease or condition in which mucin in not being produced. Examples include but are not limited to: Sjogren's syndrome, asteatosis, aging, stomatitis, and dry eye syndrome.

Diseases which are associated with mucin over- and under- production are associated with many animals. Thus, the treatments herein may be used to treat any animal which exhibits a disease associated with mucin production.

Thus, one embodiment is homologous activators or inhibitors which are specific for the animal being treated. In one embodiment, the homologs are identified by searching databases using conserved regions of the proteins. In a further embodiment, if a homolog has not been previously identified, any method known to one of skill in the art may be used to identify the animal homolog. In one embodiment, a probe or primer is used to screen for homologs in the appropriate cDNA library. The probe or primer may be designed to be degenerate, particularly in areas of the protein which are less likely to be conserved. However, typically, the probes or primers are designed to recognized more highly conserved areas of the protein.

In a further embodiment, small molecules or pharmaceuticals may be identified which inhibit the p38 MAP kinase pathway or activate the PI 3-Kinase pathway. These molecules may be identified using methods known to one of skill in the art, including high-throughput screening using the p38 MAP kinase or the PI 3-Kinase, for examples using the method of Turlais, et al. Anal. Biochem Nov. 1, 2001;298(1):62-8. In a further embodiment a phage display library is screened to identify peptides which bind to and inhibit or activate the p38 MAP kinase or the PI 3 -Kinase.

In a further embodiment, antisense oligonucleotides or TFO's are used to inhibit the mucin production by inhibiting the p38 MAP kinase pathway. Methods of identifying and producing oligonucleotides are known to those of skill in the art.

### In Hibitors of p38 MAP kinase and Activators of the PI 3-kinase Pathway

One embodiment of the invention provides for methods for inhibiting the overproduction of mucin by cells, such as middle ear cells, by applying an effective amount of at least one p38 MAP kinase inhibitor. The inhibitor may be a polypeptide, a polynucleotide, a pharmaceutical, a small molecule, or any chemical known to one of skill in the art which is pharmaceutically acceptable.

One embodiment of the invention provides for methods for inhibiting the overproduction of mucin by cells, such as middle ear cells, by applying an effective amount of at least one PI 3-Kinase activator. The activator may be a polypeptide, a polynucleotide, a pharmaceutical, a small molecule, or any chemical known to one of skill in the art which is pharmaceutically acceptable.

In one embodiment, the p38 MAP kinase inhibitor is selected from the group of chemicals consisting of: SB203580 or related compounds, including but not limited to: SB202190, SB220025, SC68376, and SKF-86002. In a further embodiment any compound developed based on the pyridinyl imidazole structure of SB203580 can be used for the inhibition of mucus overproduction. In one embodiment, the overproduction of mucin is inhibited to prevent conductive hearing loss and recurrent infection in otitis media. The inhibitor or inhibitors may be administered using any method known to one of skill in the art. In one embodiment, epithelial cells which express the inhibitor are administered to the middle ear.

In one embodiment, the inhibitor is a protein identified using the method herein which acts on the p38 MAP kinase promoter. In a further embodiment the inhibitor may be any protein which inhibits the p38 MAP kinase. In a further embodiment, the inhibitor of mucin production may be any activator of the PI 3-kinase pathway. The proteins may be used to identify homologs, variants, or truncated variants using methods known to one of skill in the art. In a further embodiment, the variants, truncated variant and homologs are at least 60% as active as the wild-type or nonmutated protein, including 65%, 70%, 75%, 80%, 85%, 90%, 95%, and 99%. In one embodiment, the variants contain single base changes which result in no change in the encodes amino acid or result in conserved changes. The mutations may occur in less conserved regions of the proteins. Alternatively the mutations are 2 or more base pair changes, or deletions or additions. The truncations, deletions, additions and base changes are more likely to appear in non-conserved regions of the protein.

In a further embodiment, the inhibitor or inhibitors are oligonucleotides. Examples of oligonucleotides which may be used for inhibition include but are not limited to: antisense oligonucleotides and TFOs (Triple helix forming oligonucleotides). In one embodiment, the oligonucleotides are administered to the cells as naked DNA. In a further embodiment, the oligonucleotides are administered as vectors which express the oligonucleotides.

### Mucin Inhibiting Composition

The proteins or active variants of the compounds herein may be purified from a natural source, such as, but not limited to, a body fluid or cells. Alternatively, they may be synthesized using methods known to one of skill in the art. Alternatively, they may be expressed recombinantly and purified by any method known to one of skill in the art. The proteins or active variants are said to be “substantially free of natural contaminants” if preparations which contain them are substantially free of materials with which these products are normally and naturally found. Active variants may be produced using methods known to those of skill in the art. However, typically, the genes coding for the proteins are cloned and mutagenesis is performed on the gene which is then expressed and the mutagenized protein isolated. Natural active variants may also be purified from a mammal which naturally produces such variants.

Compositions for use in the methods herein may contain one or more inhibitors, activators or variants selected from the group consisting of inhibitors of p38 MAP kinase, activators of PI-3 kinase, or inhibitors of Akt. In one embodiment, the composition contains only one of these proteins. In a further embodiment more than one of these proteins is included in the composition, including but not limited to two, three, and four.

In one embodiment, other treatments are included in the composition. The other treatments may be any treatments which are anti-microbial, anti-inflammatory, reduce the side-effects, enhance uptake, and increase the comfort of the patient. For example, it may be possible to include substances which reduce the drying effect on the membranes, or increase healing of the membranes in the area in which it is to be administered. For example, antibiotics may be administered or other types of antimicrobials.

### Vectors Expressing Proteins or Active Variants

It can be envisioned that one method of administering the inhibitors or activators uses expression vectors which express these proteins, peptide, or polynucleotides. The expression vectors may be targeted to the tissue or cell which is infected or which is near the infected cells. The vectors may be any vectors known to one of skill in the art including but not limited to: viral vectors, plasmid vectors, and naked DNA. Expression from these vectors may be constitutive or may be under the control of a specific promoter, such as a eukaryotic promoter, or an inducible promoter.

One advantage of using vectors for those patients who experience chronic otitis or sinusitis is that the presence of a vector may provide for longer lasting effectiveness.

### Method of Administration and Dosage

It is envisioned that the inhibitor and/or activator mixture can be administered to any type of infections which produce mucous systemically or locally. The method used may depend on the type of infection being treated. For the treatment of otitis media COME and COPD, the inhibitor and/or activator mixture may be administered locally to the ear or the sinuses or inhaled. The administration to the ear may be in a variety of ways, including, but not limited to: from the outer ear to the middle ear using a grommet, e.g. to a patient whose ear drum is pierced. Alternatively, if the infection is otitis externa, the administration may be using ear drops. If the infection is of the middle ear, the ear drops may contain a substance which allows permeabilization of the antimicrobial molecules across the ear drum. Alternatively, the inhibitor and/or activator mixture may be administered by inhalation into the lungs. In a further embodiment, the drug may be administered orally or intranasally where the mixture will act to inhibit production of mucus. Alternatively, the mixture may be administered orally, intravenously, intramuscularly, into the tear ducts, or by inhalation.

Substances which may be used to permeabilize the ear drum and allow entry of the antimicrobial molecules may include any substance which increases the permeability of membranes, such as those which are used to permeabilize skin in dermatology. Examples of such substances include, but are not limited to dimethylsulfoxide (DMSO), dimethylacetamide, methyldecyl sulfoxide, cotton seed oil, caster oil derivatives, fatty acid esters, glycerol, vesicles, liposomes, silicone vesicles (see Hill, et al. U.S. Pat. No. 5,364,633, issued Mar. 14, 1994, herein incorporated by reference), anionic surfactants, and preparations such as those in Miyazawa, et al. U.S. Pat. No. 5,500,416, issued Sep. 10, 1993 (herein incorporated by reference),

A composition is said to be “pharmacologically acceptable” if its administration can be tolerated by the recipient patient. Such an agent is said to be administered in a “therapeutically effective amount” if the amount administered is physiologically significant. Alternatively, the amount may be analyzed by the effect. For example if the chosen amount produces a reduction in the number of microbes.

The dosage of the protein components of the antimicrobial mixture to be administered may vary with the method of administration and the severity of the condition to be treated. In general, however, a dosage of from about 0.1 to 100 mg/kg/dose, and more preferably 0.5 to 50 mg/kg/dose of the drag administered 1 to 8 times a day by the intranasal route, or from 1 to 10 drops of a solution or suspension administered from 1 to 10 and preferably 1 to 6 times a day, to each ear. In a further embodiment, from about 0.01 mg/ml to about 100 mg/ml, including, but not limited to 0.1 mg/ml, 1 mg/ml, 2, mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 30 mg/ml, 40 mg/ml, 50 mg/ml, 60 mg/ml, 70 mg/ml, 80 mg/ml, and 90 mg/ml is administered to the ear, sinuses, or upper respiratory tract at least one time per day. Local administration is preferable because it reduces that chances of unwanted side-effects. However, for systemic administration, a dose of from about 0.01 mg/ml to about 1 g/ml may be administered at least one time per day for at least and including one day, and including but not limited to: 0.1 mg/ml, 0.5 mg/ml, 1.0 mg/ml, 2 mg/ml, 5 mg/ml, 10 mg/ml, 20 mg/ml, 30 mg/ml, 50 mg/ml, 75 mg/ml, 100 mg/ml, 200 mg/ml, 300 mg/ml, 500 mg/ml, 750 mg/ml, 800 mg/ml, 850 mg/ml, 900 mg/ml and 950 mg/ml.

The composition for administration may additionally include additives, excipients, thickeners, and other substances which allow for more effective administration. Examples include oils, emolients, or other substances which increase the effectiveness and comfort of ear drops, nasal sprays, and inhalable compositions. This may also include substances which enhance the smell or taste.

Additional pharmaceutical methods may be employed to control the duration. Controlled release preparations may be achieved through the use of polymers to complex or adsorb the composition. Alternatively, it is possible to entrap the composition into microcapsules, vesicles, or comparable molecules.

Selected embodiments of the method and compositions are illustrated in the Examples below:

### EXAMPLES

The following examples illustrate the mechanism of mucin production by the OM prototypic organism, NTHi. It is envisioned that similar if not identical pathways function when the infectious agent is an alternative microorganism.

In the following examples, the reagents were purchased as follows: SB203580, wortmannin, and LY294002 were purchased from Calbiochem (La Jolla, Calif.). NTHi LOS (lipooligosaccharides) was a gift from Dr. X. X. Gu (Laboratory of Immunology, National Institute on Deafness and Other Communication Disorders, NIH). Polymyxin B, lipopolysaccharides, protease inhibitor cocktail for bacterial extracts, protease E and DNase were purchased from Sigma (St. Louis, Mo.). RNase was obtained from Promega (Madison, Wis.).

The NTHi strain 12 and all other NTHi strains used in the study were clinically isolated strains that were provided by Dr. H. Faden (Children's Hospital of Buffalo, SUNY Buffalo). The strains were grown in liquid brain-heart infusion supplemented with NAD and hemin at 37° C. with 5% CO₂ as described in Shuto, et al. (2001) Proc. Natl. Acad. Sci. USA. 98, pages 8774-9 and Clemans, et al. (2000) Infect. Immun. 68, pages 4430-4440.

### EXAMPLE 1

### NTHi Up-Regulates MUC5AC Mucin Gene Transcription.

MUC5AC has been identified as a prominent mucin in respiratory secretions and in middle ear effusions of chronic otitis media with effusion (COME). To determine the role of NTHi in mucin induction, MUC5AC mRNA in human epithelial cells treated with sonicated NTHi was analyzed by RT-PCR as follows: tissue culture dishes (10 cm in diameter) were seeded with 5×10⁵ HeLa cells in a 10 ml volume of complete DMEM and incubated for 20 h. The cells were starved in serum-free medium for 18 h and then treated with or without NTHi in duplicate for 5 h. Total RNA was extracted from the lysed cells using an RNeasy minikit (Qiagen Inc., Valencia, Calif.) following the manufacturer's instructions and treated with RNase-free DNase I. cDNAs were synthesized with Moloney Murine Leukemia Virus RT (Superscript II, Life Sciences. Gaithersburg. Md.) using random hexadeoxynucleotides as primers (Promega, Madison, Wis.). After DNA synthesis, the RT was inactivated by heating the sample at 95° C. for 10 min. MUC5AC cDNA was amplified with primers 5′-TCC GGC CTC ATC TTC TCC-3′ (SEQ ID NO:1) and 5′-ACT TGG GCA CTG GTG CTG-3′ (SEQ ID NO:2) and cyclophilin was amplified with 5′-CCG TGT TCT TCG ACA TTG CC-3 ′ (SEQ ID NO:3) and 5′-ACA CCA CAT GCT TGC CAT CC-3′ (SEQ ID NO:4). PCR was performed for 15 min at 95° C., 1 min at 94° C., 1 min at 57° C. (50° C. for cyclophilin) and 1 min at 72° C. for each cycle and 7 min at 72° C. after all of the cycles. A cycle number that was in the linear range of amplification was selected for PCR analysis; 32 cycles for MUC5AC and 26 for cyclophilin.

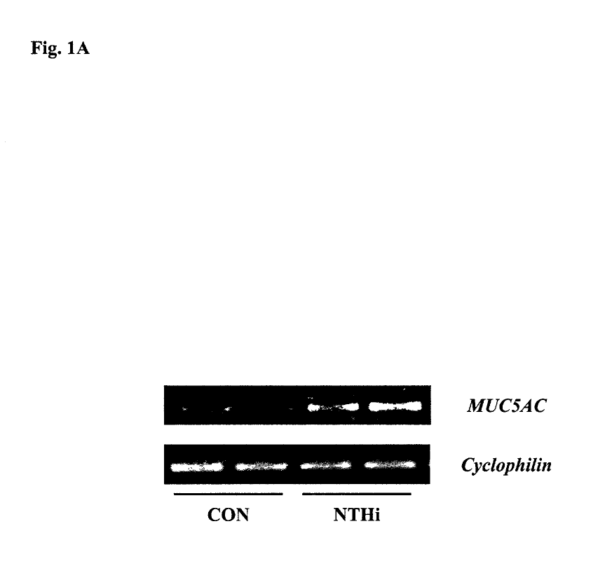

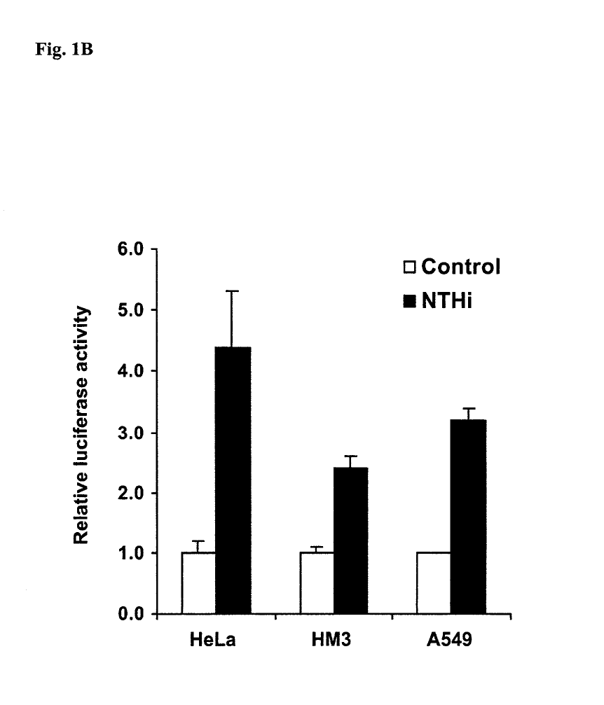

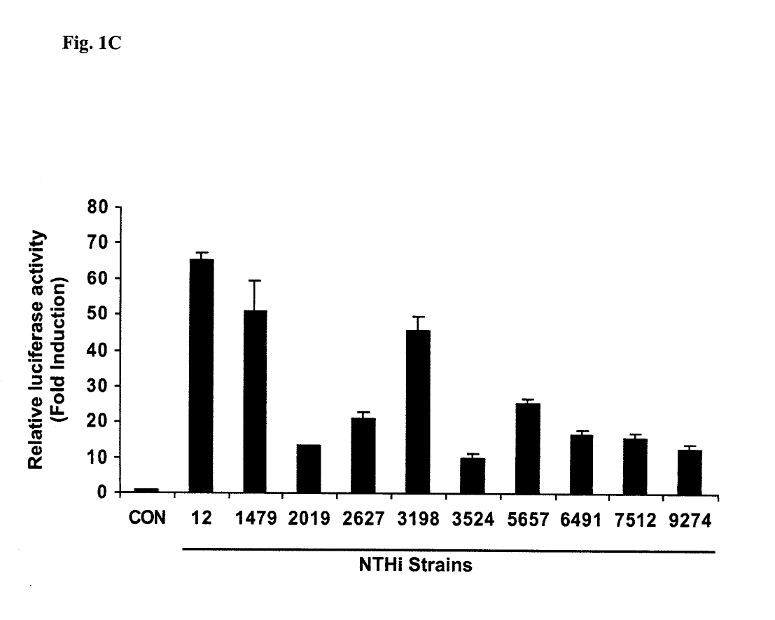

As shown in FIG. 1A, MUC5AC mRNA levels significantly increased when the cells were treated with NTHi for 5 h. To investigate whether transcriptional regulation was involved in MUC5AC induction, human epithelial cells including HeLa, HM3 and A549 were transfected with a MUC5AC promoter-luciferase reporter construct and treated with NTHi. The HeLa (human cervix epithelial) cells were cultured in MEM. HM3 (human colon epithelial) cells were maintained in DMEM. A549 (human lung epithelial) cells were maintained in F-12 Nutrition Mixture (Kaighn's Modification). All media contained 10% fetal bovine serum (Gibco-BRL), penicillin (100 units/ml) and streptomycin (0.1 mg/ml). All cells were cultured in a humidified atmosphere of 5% CO₂/95% air.

The luciferase activity driven by the MUC5AC promoter indeed increased upon exposure to NTHi, suggesting that transcriptional regulation may be involved (FIG. 1B). To further determine whether other clinical isolates of NTHi strains could also upregulate MUC5AC, a variety of NTHi clinical isolates were tested for MUC5AC-inducing activity. Interestingly, all clinical isolates tested were capable of inducing MUC5AC although their mucin-inducing activity differed quantitatively (FIG. 1C). This result suggests that the mucin-inducing activity of NTHi is well conserved among all ten strains that were tested. Strain 12, the strain with the most potent MUC5AC-inducing activity, was used for further investigations.

In example 2 the bacterial components necessary for induction of mucin production were identified.

### EXAMPLE 2

### Cytoplasmic Components of NTHi Play a Major Role in MUC5AC Induction.

Having demonstrated that NTHi up-regulates MUC5AC transcription, determination of the bacterial components responsible for MUC5AC induction was next addressed. Based on the fact that there was a dramatic increase in COME cases after antibiotic was introduced as a treatment for otitis media, it appeared that bacterial breakdown components released from lysed bacteria may have played an important role in mucin induction. To test this hypothesis, NTHi bacteria were first disrupted by sonication; the mucin-inducing activity of sonicated NTHi was then tested using MUC5AC promoter luciferase assay.

The cytoplasmic components were isolated as follows: the bacterial cells were harvested when they reached middle to late log phase and resuspended in PBS with the same volume (1×) or ⅓ of the original volume (3×). The bacterial cell suspension was sonicated on ice three times at 150 Watts for 3 min with 5 min intervals between each sonication. Residual cells were removed by centrifugation (10,000×g, 4° C. 10 min). Cytoplasmic components were obtained from the supernatant of sonicated bacteria by ultracentrifugation (1,000,000×g, 4° C., 1 h), and stored at −80° C.

The luciferase assay was performed as follows: expression plasmids fp38α(AF) and fp38β(AF) were previously described in Shuto, et al. (2001) Proc. Natl, Acad. Sci. U.S.A. 98, pages 8774-9. The expression plasmids p110, p85α, Akt KD, and Akt WT were provided by D. Stokoe (University of California, San Francisco). The reporter construct MUC5AC contained 3.7-kb 5′-flanking region of the human MUC5AC mucin gene in a luciferase reporter vector pGL3 (Li, et al. 1998, J. Biol. Chem. 273, 68126820). Transient transfections of cells were performed in triplicate with Trans IT-LT1 (Panvera, Madison, WT) following the manufacturer's instructions. Forty-two hours after transfection, the cells were treated with NTHi for 4 h and then harvested for use in the luciferase assay. For experiments with inhibitors, HM3 cells stably transfected with MUC5AC-luciferase plasmid were pretreated with inhibitors for 1-2 h, then treated with NTHi for 4 h and harvested for luciferase assays. Luciferase assays were performed on a Monolight 3010 luminometer for 15 s (Analytical Luminescence, San Diego, Calif.). The NTHi-dependent fold induction was calculated relative to the luciferase light units obtained in the absence of NTHi treatment. The normalized luciferase activity was thus expressed as relative luciferase activity (fold induction).

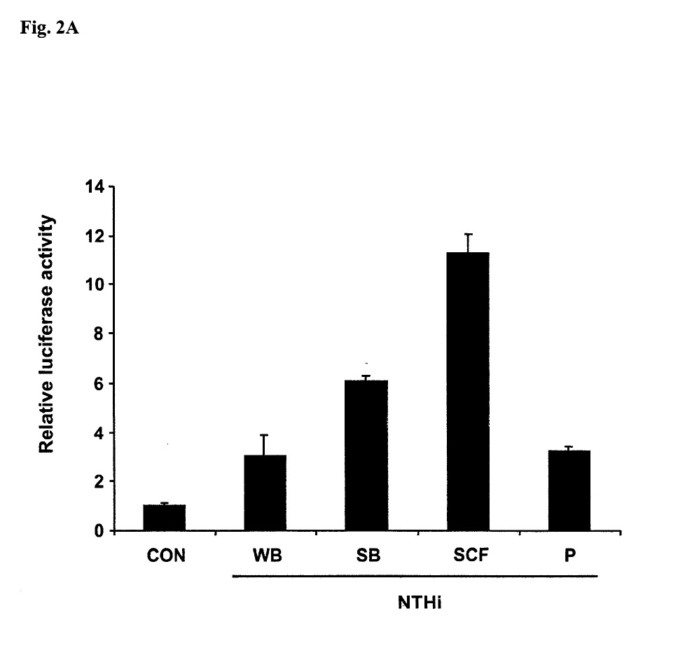

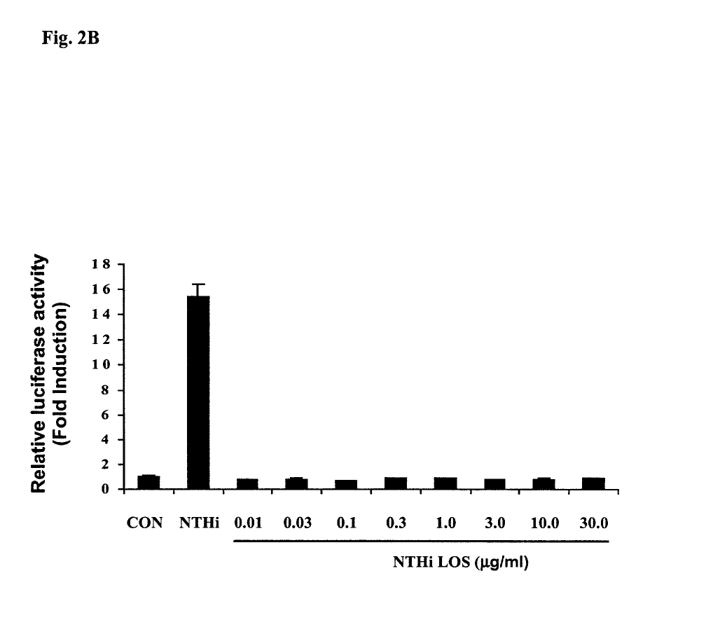

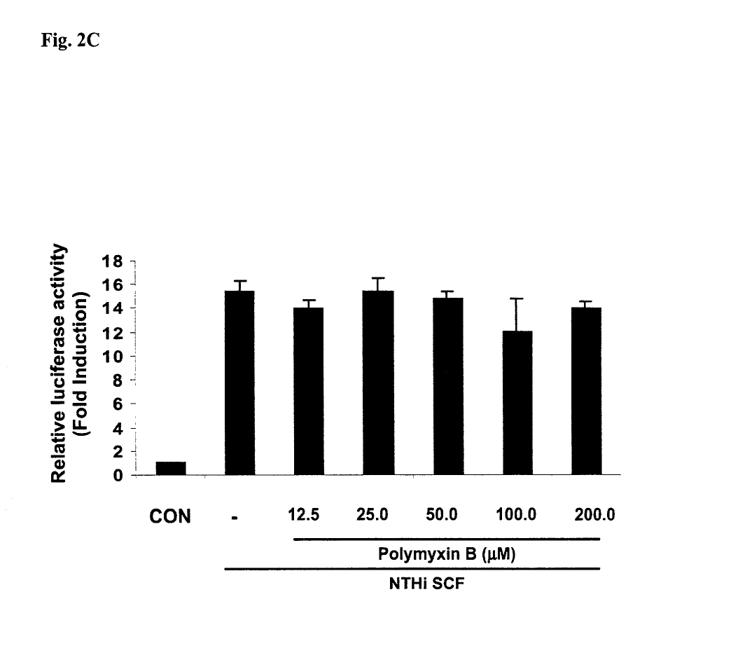

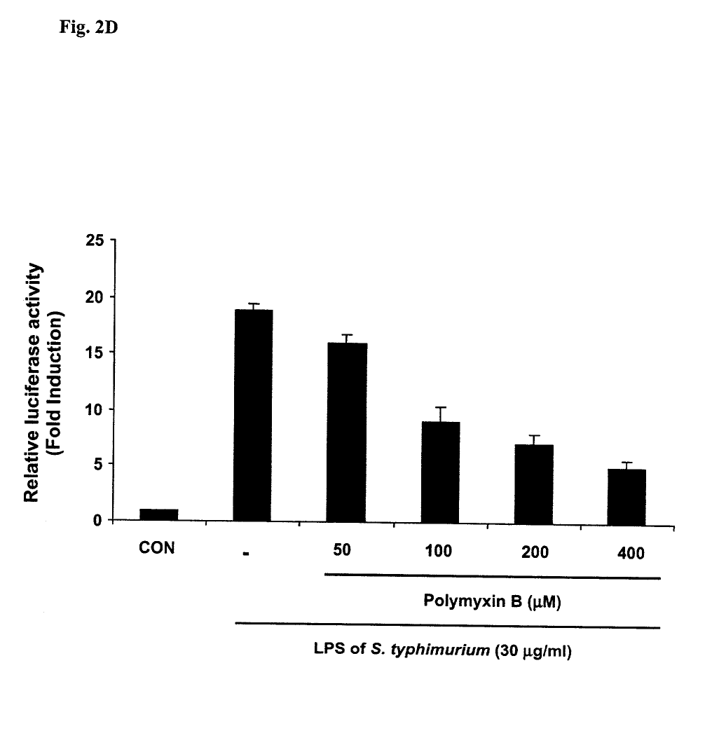

As shown in FIG. 2A, NTHi whole bacteria (WB) induced modest levels of MUC5AC transcription. However, the mucin-inducing activity was greatly increased when NTHi bacteria were sonicated (SB), indicating that bacterial cell lysis by sonication released additional potent mucin inducers. To determine which fraction of sonicated NTHi lysate was responsible for the greatly increased activity, the sonicated bacterial lysate was further separated by centrifugation into a pellet (P), which contained membrane debris as well as residual whole cells, and soluble cytoplasmic fractions (SCF). The SCF fraction was even more potent than sonicated bacterial lysate while the activity in the pellet was low, similar to that in non-sonicated whole bacteria. Because many bacteria are capable of secreting bioactive molecules into the environment, the possibility that NTHi produced diffusible mucin inducers was evaluated. No significant mucin-inducing activity was detected in bacterial culture supernatant, suggesting that mucin inducers were not secreted by live intact bacteria.

Previous results by the Applicants showed that lipopolysaccharide (LPS) from gram-negative bacteria P. aeruginosa up-regulated MUC2 mucin transcription. Like other gram-negative bacteria, NTHi also contains lipooligosaccharide (LOS), although its LOS differs from LPS in other gram-negative bacteria in a number of ways including the number of O-side chains. NTHi LOS has been shown to induce cytokine expression in epithelial cells. Because the NTHi cytoplasmic components may contain LOS, it was of interest to determine whether LOS was involved in MUC5AC induction. When transfected epithelial cells were treated with LOS, no mucin-induction was detected (FIG. 2B). To corroborate this, the soluble cytoplasmic fraction (SCF) was ultracentrifuged to further spin out bacterial envelope debris and was then pretreated with various concentrations of polymyxin B, which binds LOS and would neutralize the biological activity of any remaining LOS. As shown in FIG. 2C, no significant reduction in NTHi-induced MUC5AC transcription occurred after polymyxin B treatment. Importantly, the potency of the polymyxin B was shown by the fact that it significantly reduced MUC5AC transcription induced by LPS from S. typhimurium (FIG. 2D). These data indicate that, unlike NTHi induction of inflammatory cytokines and P. aeruginosa induction of MUC2, NTHi induction of MUC5AC does not require LOS.

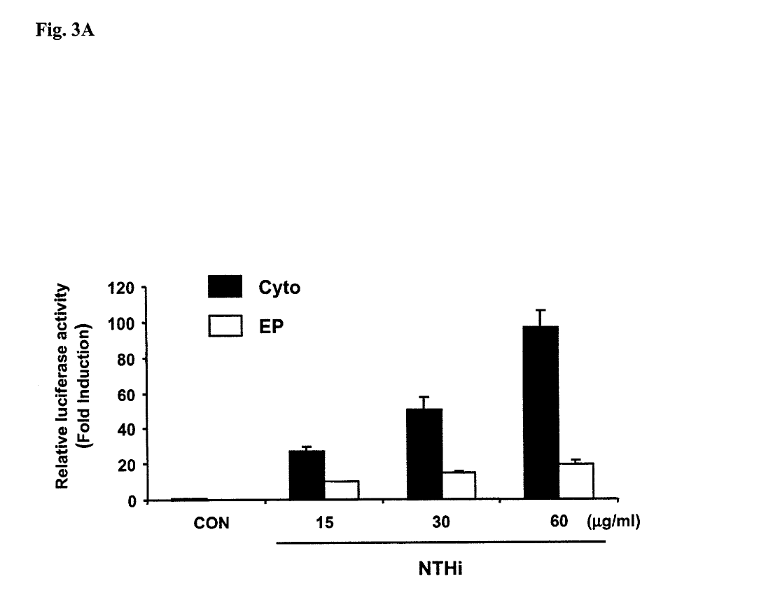

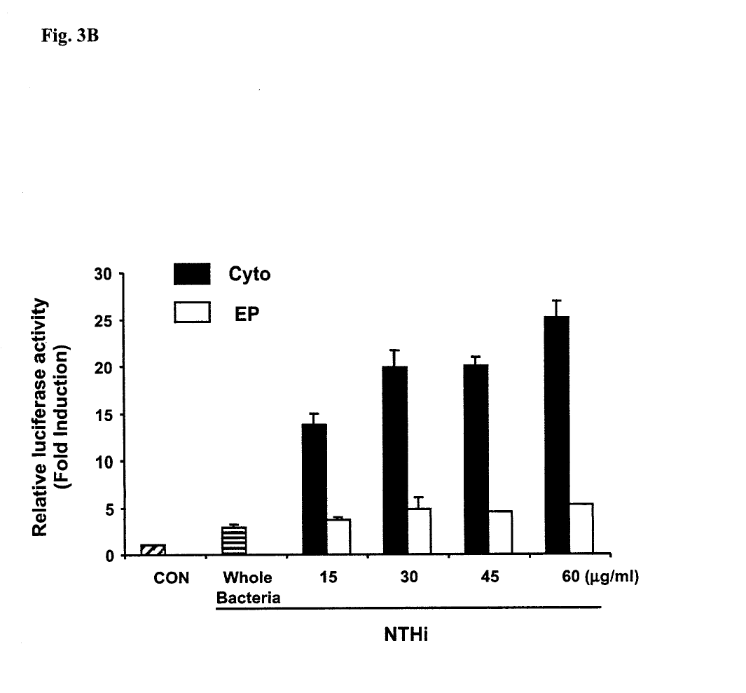

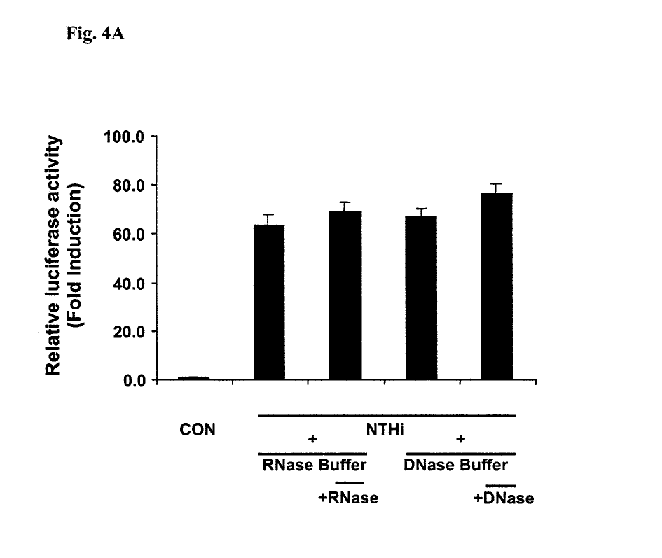

In addition to LOS, NTHi surface membrane proteins have also been shown to play an important role in the pathogenesis of NTHi infections. To determine whether NTHi membrane proteins played an important role in the MUC5AC induction, equivalent amounts of envelope proteins (EP) and cytoplasmic components (Cyto) were compared for their mucin inducing activity. As shown in FIG. 3A, NTHi cytoplasmic components induced MUC5AC transcription to a much greater degree than envelope proteins. To further verify that the MUC5AC-inducing activity indeed resided in the cytoplasmic fraction rather than being due to an effect of sonication on membrane proteins, the bacteria were disrupted in a French Pressure cell, which has been commonly used as an alternative way to completely disrupt bacteria. The cytoplasmic components were separated from the envelope proteins using centrifugation and their MUC5AC-inducing activity was then assessed. Consistent with the envelope and cytoplasmic components prepared by sonication, NTHi cytoplasmic components prepared using French Pressurecell also strongly up-regulated MUC5AC transcription whereas the whole bacteria and membrane proteins induced MUC5AC up-regulation to a much lesser extent (FIG. 3B). Therefore, cytoplasmic components of NTHi play a major role in NTHi-induced MUC5AC transcription.

The unexpected finding of the negative effect of NTHi LOS on MUC5AC transcription is interesting. While LPS from other gram-negative bacteria such as Pseudomonas aeruginosa (P. aeruginosa) and Salmonella typhimurium (S. typhimurium) up-regulates MUC2 and MUC5AC transcription, LOS did not. Additionally, induction of proinflammatory cytokines by NTHi LOS has also been reported. Based on these studies, a stimulating effect of LOS on MUC5AC was expected. The negative effect shown in FIG. 2B and 2C is unexpected, because it was in sharp contrast to the up-regulation of mucin by LPS from S. typhimurium and P. aeruginosa. However, the structures of LPS and LOS have difference. In comparison with LPS, LOS lacks an O-specific polysaccharide. Therefore it seemed logical that this structural difference may account for the negative effect on MUC5AC induction. However, this notion is not supported by the fact that LPS molecules purified from a polysaccharide-deficient strain and a wild-type strain of P. aeruginosa were equipotent in induction of MUC2, suggesting that lipid A and the sugar core region are sufficient for mucin induction. In view of the structure of other regions, LOS also differs from LPS in the structure of the lipid A component. A previous antigenic analysis of NTHi lipid A showed that a monoclonal antibody specific for the lipid A portion of NTHi LOS recognized the lipid A determinant on most NTHi strains but did not recognize the lipid A of 39 stains from 14 non-Haemophilus influenzae species. Thus, differences in the lipid A region between NTHi LOS and other bacterial LPS may alternatively or also be responsible for the difference in mucin induction. Although no direct up-regulation of MUC5AC by NTHi LOS was shown in vitro, the data do not preclude the possibility that LOS may indirectly up-regulate MUC5AC in vivo by inducing cytokines such as TNF-α, which has been shown to up-regulate mucin.

### EXAMPLE 3

### Proteins are the Major NTHi Cytoplasmic Components Responsible for MUC5AC Induction.

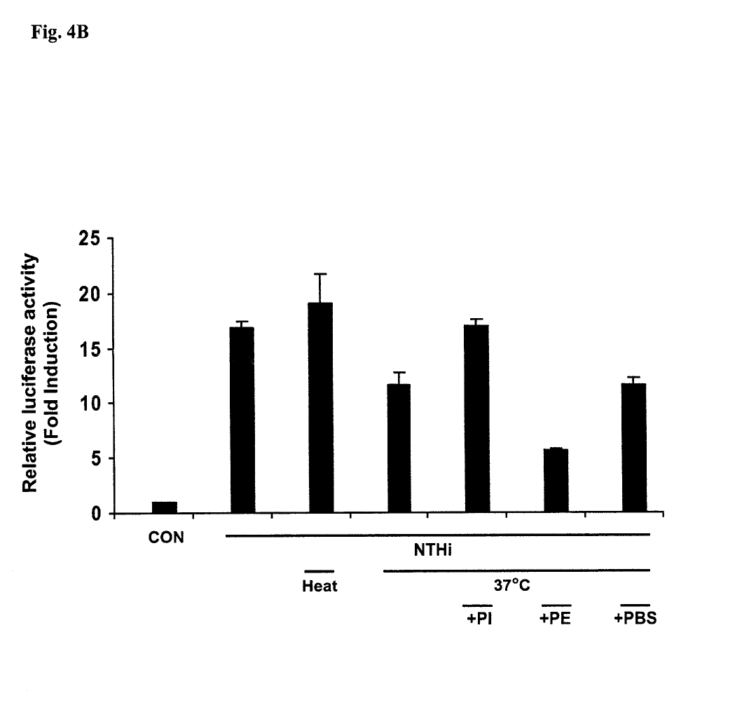

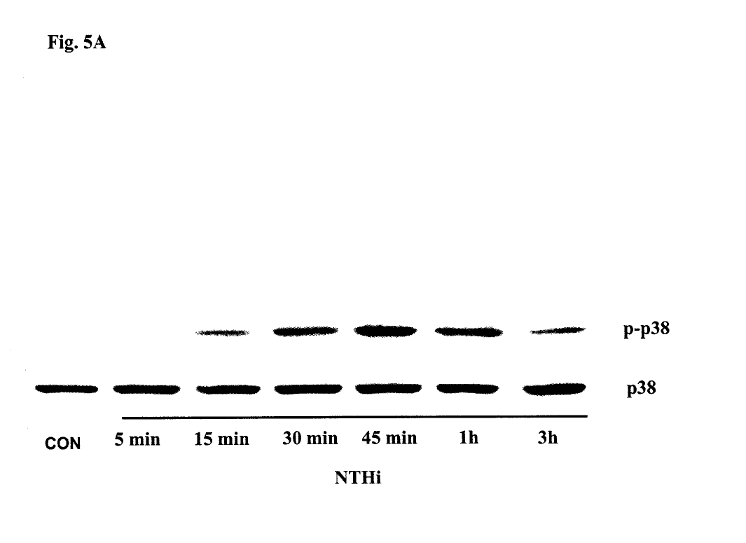

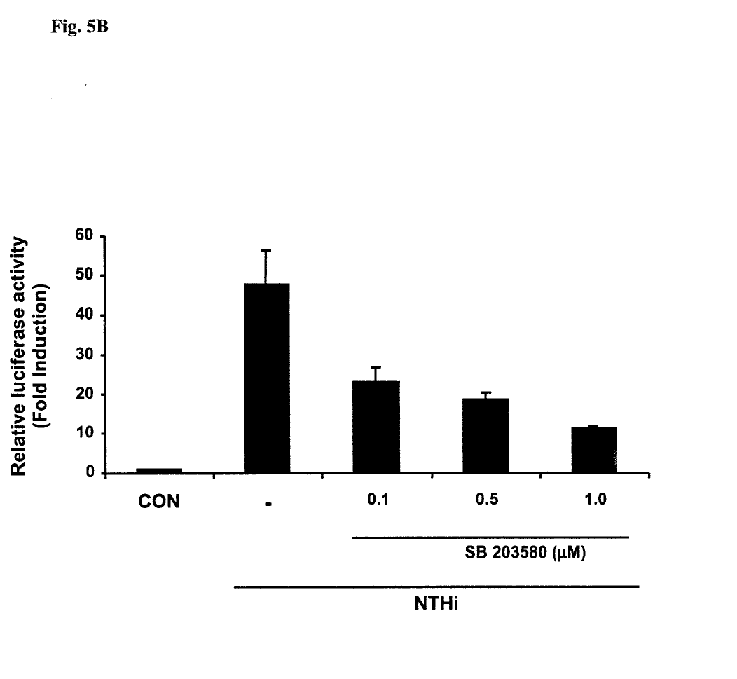

The NTHi cytoplasmic content is a complex mixture containing mainly nucleic acids and proteins. In an effort to better define the mucin inducer, the cytoplasmic fraction was first pretreated with DNase or RNase. Complete digestion of nucleic acids was confirmed by electrophoresis. As shown in FIG. 4A, neither DNase nor RNase reduced MUC5AC induction, demonstrating that nucleic acids are not involved. The cytoplasmic components were also heated at 100° C. for 5 min, or kept at 37° C. overnight. The results in FIG. 4B showed that 100° C., a denaturing temperature, did not have any effect on the MUC5AC-inducing activity whereas overnight incubation at 37° C. reduced the activity. To determine whether the reduced activity following overnight incubation at 37° C. might be caused by endogenous proteases in the cytoplasmic fraction, bacterial protease inhibitors (PI) were added. The addition of PI to the cytoplasmic fraction counteracted the reduction in activity, indicating that proteins in the NTHi cytoplasm are responsible for mucin induction. This was confirmed by treatment of the cytoplasmic fraction with an exogenous protease. Mucin-inducing activity was reduced after the cytoplasmic fraction was treated at 37° C. overnight. When the same fraction was incubated with protease E (PE) for another 2 h, the activity was further substantially reduced. Thus, heat-stable NTHi cytoplasmic proteins play a major role in NTHi-induced MUC5AC transcription.

### EXAMPLE 4

### Activation of p38 MAP kinase is Required for NTHi-Induced MUC5AC Transcription.

Having identified cytoplasmic proteins as major inducers of MUC5AC transcription by NTHi, the intracellular signaling pathways which were involved had not been identified. Among numerous host signaling pathways, MAP kinase (mitogen-activated protein kinase) pathways play a key role in variety of cellular responses. p38, a major MAP kinase superfamily member, has been shown to be involved in NTHi-induced inflammatory responses. Thus, to determine the role of p38 in NTHi-induced MUC5AC up-regulation, NTHi cytoplasmic proteins were investigated for the ability to activate p38 MAP kinase. Phosphorylation of p38 MAP kinase was determined by Western blot analysis using antiphosphorylated p38 MAP kinase antibody as follows: HeLa and HM3 cells were treated with or without NTHi. Total cell lysates were analyzed by antibodies against phospho-p38 (Thr180/182), p38, phospho-Akt (Ser473) and Akt (New England Biolabs, Beverly, Mass.) as described following the manufacturer's instructions.

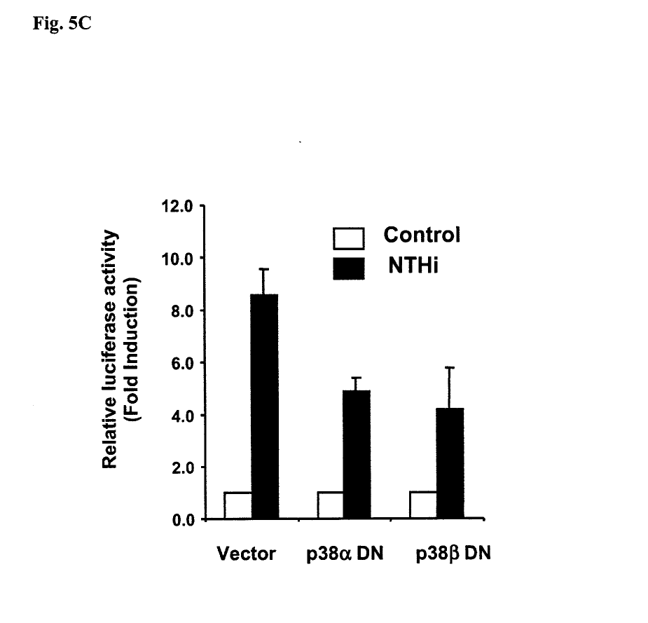

FIG. 5A. shows phosphorylation of p38 MAP kinase in HM3 cells treated with NTHi cytoplasmic proteins for various times. The p38 phosphorylation appeared at 15 min, peaked at 45 min and declined thereafter. These results indicated that NTHi strongly activates p38 MAP kinase. It was next of interest to determine whether activation of p38 MAP kinase was required for MUC5AC induction. As shown in FIG. 5B, the pyridinyl imidazole SB203580, a specific chemical inhibitor for p38 MAP kinase, inhibited MUC5AC induction in response to NTHi cytoplasmic proteins in a dose-dependent manner. To confirm the involvement of p38 MAP kinase, the cells were co-transfected with a MUC5AC-luciferase reporter construct and a dominant-negative mutant of either p38α or p38β. The MUC5AC-inducing activity was inhibited by the dominant-negative mutants of both p38α and p38β (FIG. 5C.). Thus, activation of both p38 α and β is involved in MUC5AC induction by NTHi cytoplasmic proteins.

### EXAMPLE 5

### Inhibitors for PI 3-kinase Markedly Enhance Mucin Induction

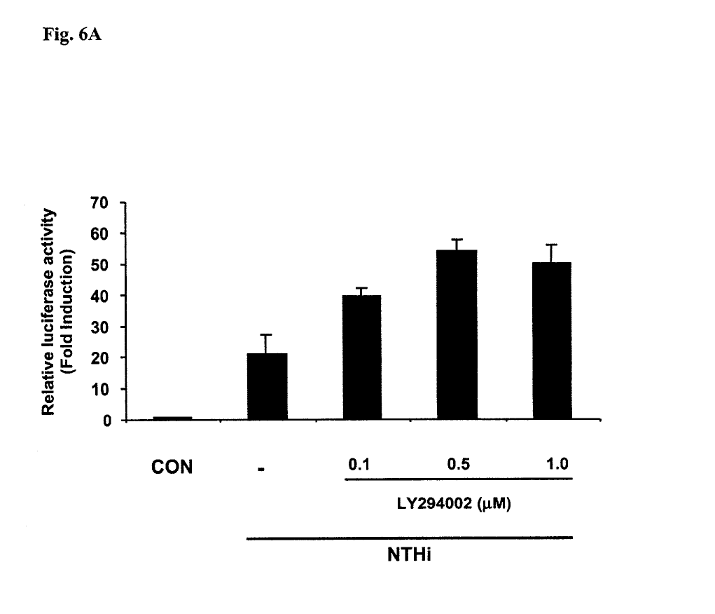

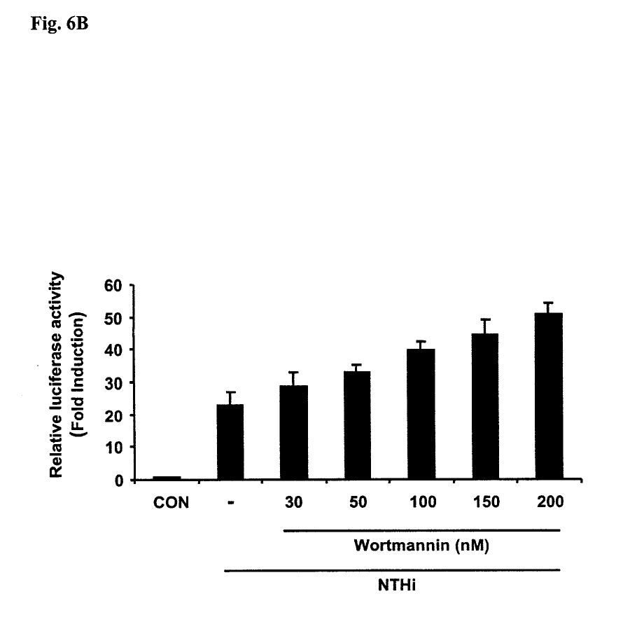

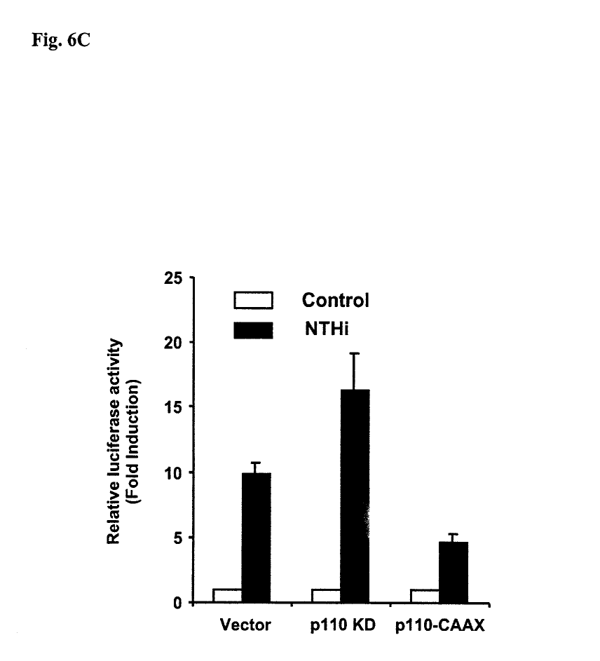

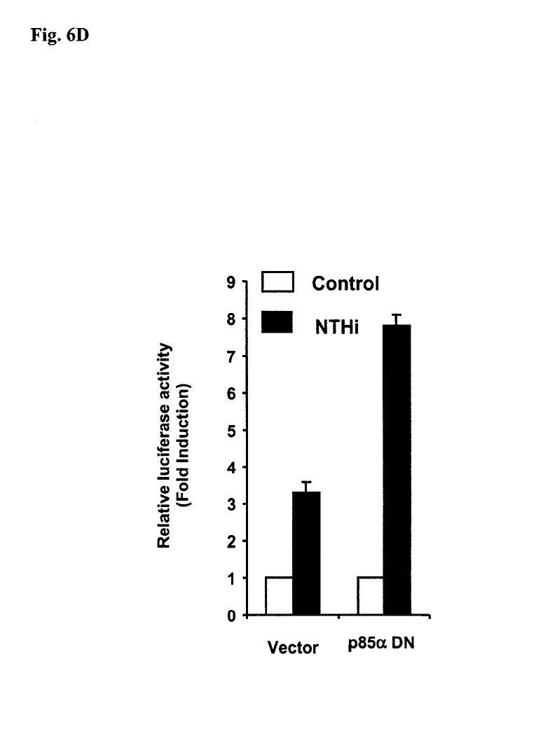

In addition to p38 MAP kinase, phosphoinositide 3-kinase (PI 3-kinase) represents another major signaling transducer involved in a variety of cellular responses. It is a heterodimer consisting of p85, the regulatory subunit, and p110, the catalytic subunit. Activation of PI 3-kinase catalyses the phosphorylation of phosphatidylinositol. The phosphorylated lipids bind to Akt, a serine-threonine kinase, resulting in membrane localization and a conformational change of Akt. This allows Akt to be phosphorylated and activated to mediate a variety of cellular responses such as protection of cells from apoptosis and induction of NF-κB. There is also evidence that PI 3-kinase is involved in bacterial pathogenesis. Because of the importance of PI 3-kinase in cellular responses as well as in bacterial pathogenesis, it was of interest to determine the potential involvement of PI 3-kinase in NTHi-induced MUC5AC transcription. The effects of LY294002 and wortmannin, specific inhibitors for PI 3-kinase, on MUC5AC induction were examined. Surprisingly, both inhibitors markedly enhanced the MUC5AC induction in a dose-dependent manner (FIG. 6A. and 6B.), suggesting that activation of PI 3-kinase was negatively involved in NTHi-induced MUC5AC transcription. To confirm this, HeLa cells were co-transfected with the MUC5AC-luciferase reporter plasmid and either dominant-negative mutants or a constitutively active form of PI 3-kinase, then treated with NTHi. Consistent with the effects of the chemical inhibitors, overexpression of the dominant-negative mutant forms of p110 (p110 KD) and p85 (p85α DN) significantly enhanced, whereas overexpression of the constitutively active form of p110 (p110-CAAX) reduced, MUC5AC induction by NTHi (FIG. 6C and 6D).

### EXAMPLE 6

### Identification of the Downstream Target of PI 3-kinase

Next, the downstream target of PI 3-kinase involved in NTHi-induced MUC5AC transcription was identified. Because Akt represents one of the most important signaling molecules downstream of PI 3-kinase, it was a likely candidate as a target of PI 3-kinase. Western Blot analysis was performed to determine whether NTHi activates Akt as follows: HeLa and HM3 cells were treated with or without NTHi. Total cell lysates were analyzed by antibodies against phospho-p38 (Thr180/182), p38, phospho-Akt (Ser473) and Akt (New England Biolabs, Beverly, Mass.) as described following the manufacturer's instructions.

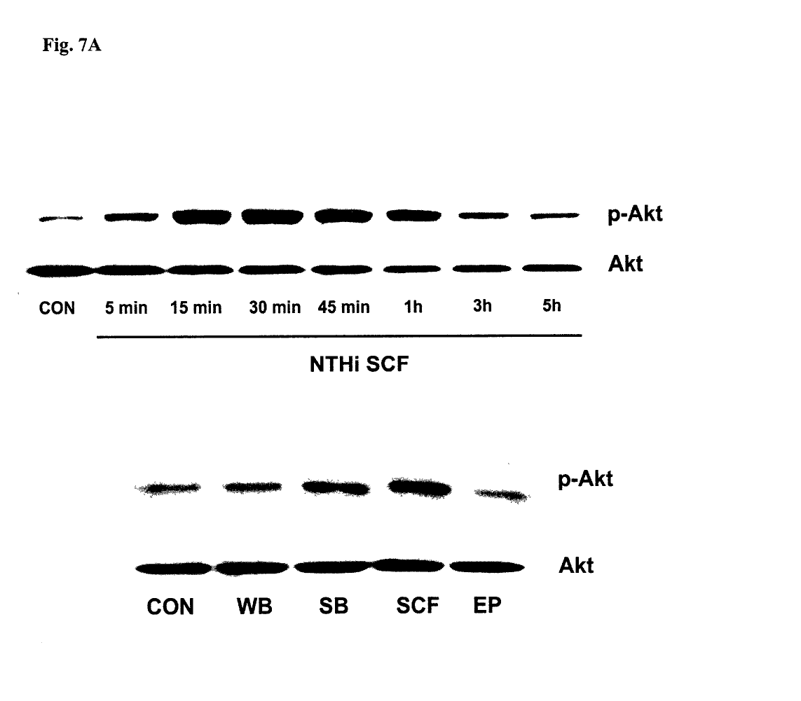

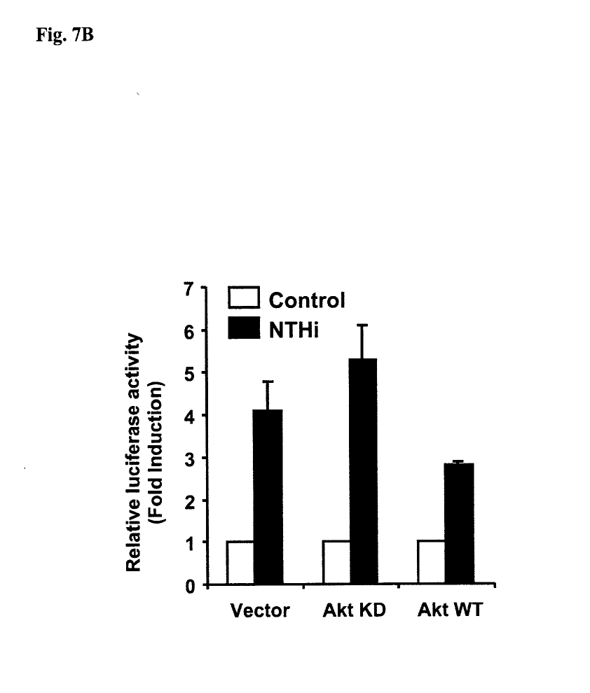

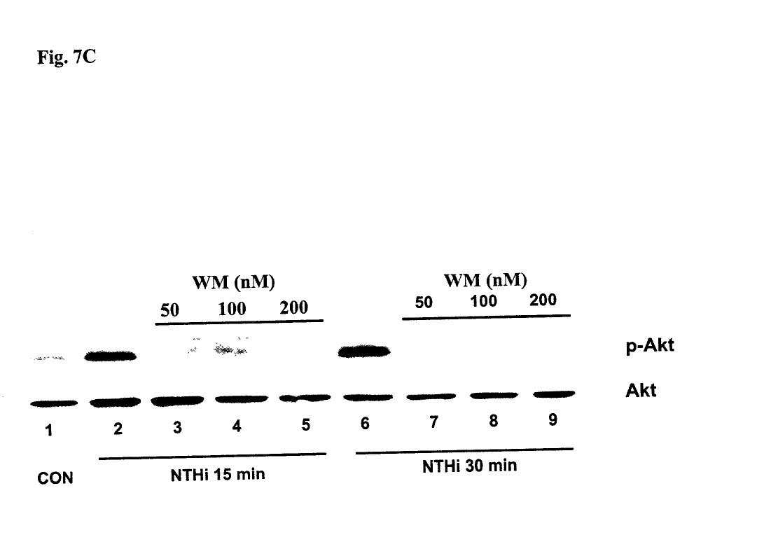

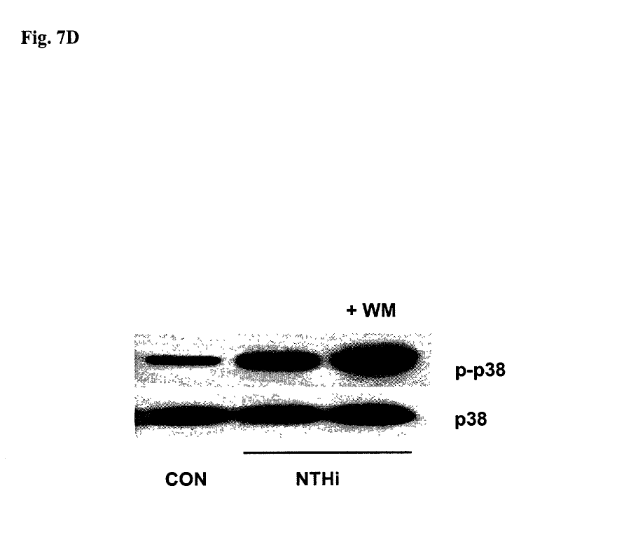

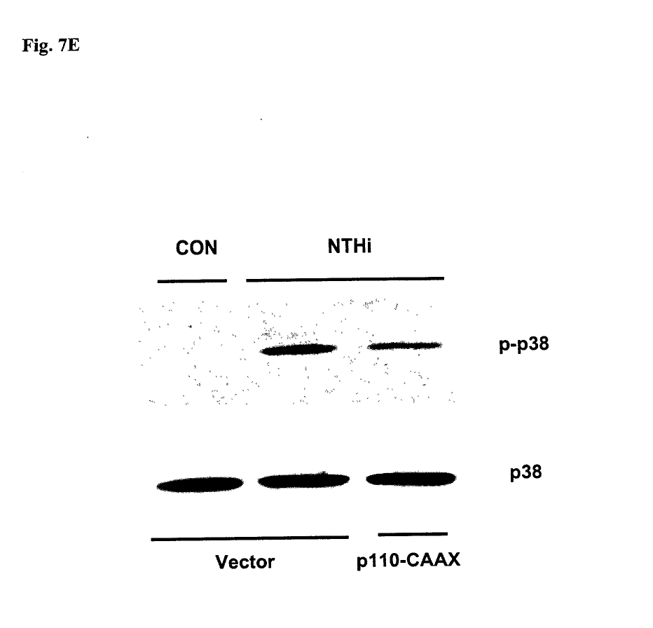

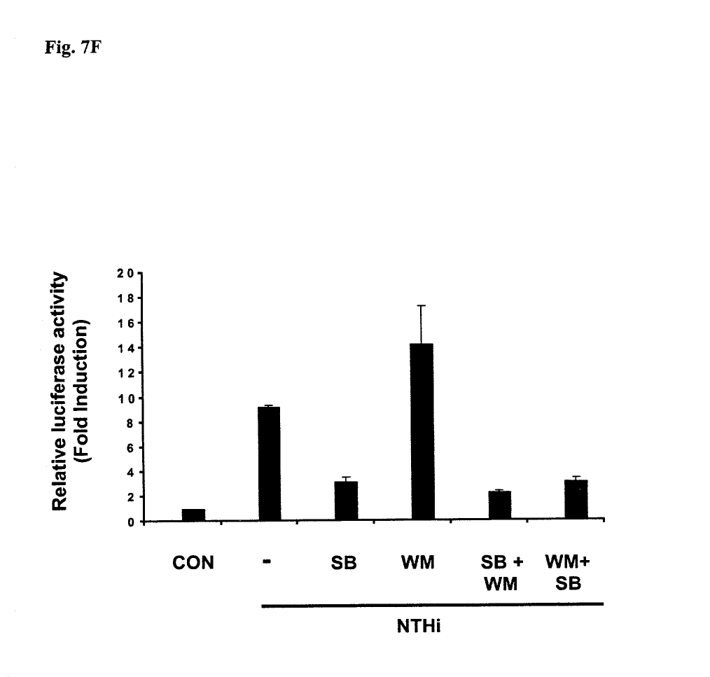

As shown in FIG. 7A (upper panel), phosphorylation of Akt significantly increased after 5 min of treatment with NTHi SCF. The phosphorylation of Akt peaked at 30 min and then declined to the basal level at 5 h after treatment. This finding suggests that, in addition to p38, NTHi SCF also activates Akt. Since, as shown in FIG. 2A, other NTHi fractions are also capable of inducing MUC5AC transcription, it was of interest to test these fractions for their ability to activate Akt. Interestingly, all treatments including the whole bacteria induced Akt phosphorylation although their Akt-inducing activity differed quantitatively (FIG. 7A, lower panel). It was next determined whether Akt was involved in NTHi-induced MUC5AC transcription. As shown in FIG. 7B, overexpression of a dominant-negative mutant of Akt (Akt KD) enhanced, whereas overexpression of a wild-type of Akt (Akt WT) reduced, the MUC5AC induction. These results indicate that Akt is also negatively involved in NTHi-induced MUC5AC transcription. Since PI 3-kinase is not the only upstream kinase of Akt, the effect of wortmannin on NTHi-induced Akt phosphorylation was next determined to establish the link between the PI 3-kinase and Akt. As shown in FIG. 7C, wortmannin abrogated Akt phosphorylation induced by NTHi cytoplasmic proteins, indicating that Akt indeed acts downstream of PI 3-kinase in response to NTHi.

### EXAMPLE 7

### Phosphoinositide 3-kinase (PI 3-kinase)-Akt Signaling Pathway is Negatively Involved in the NTHi-Induced MUC5AC Transcription via a Negative Cross-Talk With p38 MAP kinase

Having identified p38 MAP kinase as a positive pathway and PI 3-kinase-Akt as a negative pathway involved in NTHi-induced MUC5AC transcription, still unknown was whether or not there was a negative cross-talk between these two signaling pathways. Based on a recent report that inhibition of PI 3-kinase-Akt signaling led to enhanced VEGF activation of p38 MAP kinase, the effect of wortmannin on the phosphorylation state of p38 MAP kinase induced by NTHi was next determined. FIG. 7D shows that pretreatment of HM3 cells with wortmannin greatly enhanced phosphorylation of p38 induced by NTHi. To determine whether the activation of PI 3-kinase-Akt pathway may lead to down-regulation of NTHi-induced p38 MAP kinase phosphorylation, an activated, membrane-targeted form of p110 (p110-CAAX) was transfected into HM3 cells. As shown in FIG. 7E, NTHi-induced phosphorylation of p38 MAP kinase was attenuated by overexpression of p110-CAAX, indicating that activation of PI 3-kinase-Akt indeed led to down-regulation of p38 MAP kinase phosphorylation induced by NTHi. To further determine whether PI 3-kinase-Akt pathway could bypass the p38 MAP kinase pathway to down-regulate MUC5AC transcription, the cells were first pretreated with SB203580, a specific inhibitor for p38 MAP kinase and then the cells were pretreated with wortmannin, a specific inhibitor for PI 3-kinase, or vice versa, before NTHi was added to the cells. As shown in FIG. 7F, wortmannin no longer enhanced NTHi-induced MUC5AC transcription in the cells that were already pretreated with SB203580, whereas SB203580 was still capable of inhibiting NTHi-induced MUC5AC transcription in the cells that were already pretreated with wortmannin. Taken together, these results demonstrated that activation of PI3-kinase-Akt signaling pathway leads to attenuation of p38 MAP kinase phosphorylation. Thus, PI 3-kinase-Akt served as an inhibitory signaling pathway in NTHi-induced MUC5AC transcription via a negative cross-talk with p38 MAP kinase pathway.

### EXAMPLE 8

### The Involvement of Autolysis in Mucin Production by NTHi

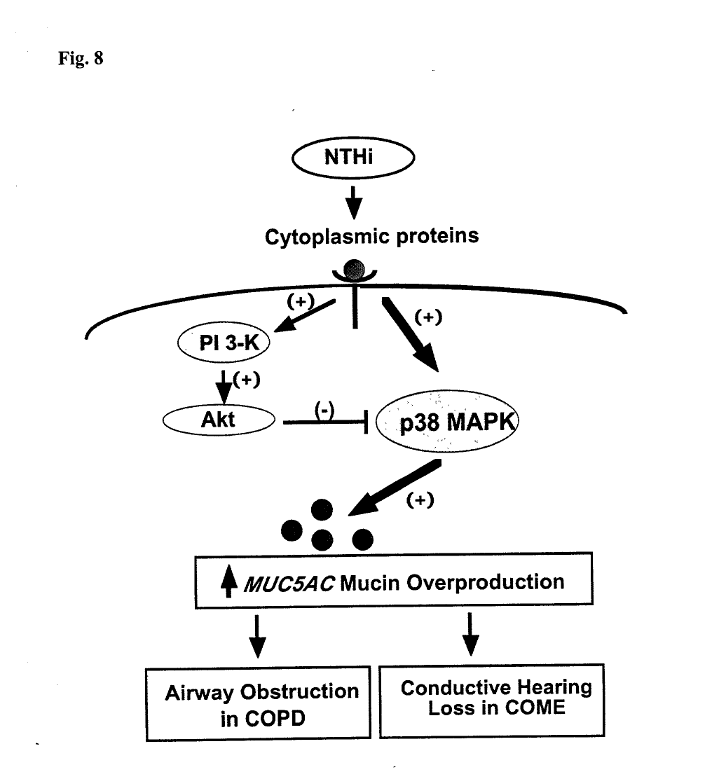

In the present study, the involvement and mechanism of NTHi in the up-regulation of MUC5AC mucin gene transcription in human epithelial cells was determined. Here, we show that NTHi cytoplasmic proteins up-regulate MUC5AC transcription via a positive p38 MAP kinase signaling pathway and a negative PI 3-kinase-Akt signaling pathway (FIG. 8).

A major finding was the experimental evidence for the involvement of bacterial cytoplasmic proteins in MUC5AC induction. This result, although rather unexpected, may well explain why many patients still have persistent symptoms such as middle ear effusion in COME even after intensive treatment with antibiotics. One of the major characteristics of NTHi is its tendency to autolyse. Its autolysis can be triggered in vitro when the bacteria culture is old, and in vivo under various conditions including antibiotic treatment. Clinical microbiology studies have shown that most effusions from the patients with COME did not contain viable bacteria when cultured, whereas bacterial DNA could be detected by PCR in 80% of effusions, often in the absence of viable bacteria on culture. In addition, previous results have shown that endotoxin was present in 67% of middle ear effusions that were negative as determined by culture for any bacterium. Despite some potential underestimation of the prevalence of viable bacteria by conventional culture, these results clearly indicated that bacterial breakdown products or components released from lysed bacteria persist in the middle ear even after bacteria die and thus may act as long lasting stimuli of mucin production and inflammatory responses. Thus, the cytoplasmic proteins released from the lysed NTHi bacteria after treatment with antibiotics may contribute substantially to the pathogenesis of otitis media by directly up-regulating MUC5AC mucin transcription.

In the present study, evidence is provided for the first time that activation of p38 MAP kinase is required for up-regulation of MUC5AC by NTHi cytoplasmic protein(s). In addition, the PI 3-kinase-Akt signaling pathway is also activated by NTHi, which, however, leads to down-regulation of p38 MAP kinase activity. Negative cross-talk has been established by previous studies between PI 3-kinase-Akt pathway and MAP kinases including the extracellular signal-regulated kinases (ERK) and the c-jun NH2-terminal kinase (JNK). Whether or not there is also negative interaction between PI 3-kinase-Akt and p38 MAP kinase has remained unclear. Recently, a report by Gratton et al. showed that blockade of PI 3-kinase-Akt led to enhanced vascular endothelial growth factor (VEGF) activation of p38 MAP kinase. However, little was known about the involvement of this negative cross-talk in bacterial pathogenesis as well as in mucin gene regulation. In the present study, PI 3-kinase-Akt was found to serve as an inhibitory signaling pathway in NTH-induced MUC5AC transcription via a negative cross-talk with p38 MAP kinase. Although inhibition of PI 3-kinase-Akt signaling by wortmannin enhanced, whereas activation of PI 3-kinase-Akt by overexpression of an activated form of p110 attenuated, NTHi-induced activation of p38 MAP kinase, the possibility that PI 3-kinase-Akt pathway may interact with the upstream kinases of p38 MAP kinases such as MAP kinase kinase 3 and 6 (MKK3/6) can not be ruled out. It is also unclear whether a direct physical interaction between PI 3-kinase-Akt and MKK3/6-p38 MAP kinase is involved in this cross talk.

Although the present invention has been described in terms of certain preferred embodiments, other embodiments of the invention will become apparent to those of skill in the art in view of the disclosure herein. Thus, obvious changes and modifications may be made without departing from the spirit and scope of the invention. Accordingly, the scope of the invention is not intended to be limited by the foregoing, but rather to be defined only by the claims which follow.

What is claimed is:

1. A method for the treatment of overproduction of mucin in a mammal, comprising:
administering an inhibitor of p38 MAP kinase to the mammal in an amount sufficient to reduce mucin production

2. The method of claim 1 wherein the overproduction of mucin is caused by an otitis media (OM) infection or chronic obstructive pulmonary disease (COPD).

3. The method of claim 2 wherein the OM or COPD is caused by nontypeable Haemophilus influenzae (NTHi).

4. The method of claim 1 wherein said inhibitor of p38 MAP kinase is a chemical inhibitor selected from the group consisting of: pyridimylimidzol SB203580, SB202190, SB220025, SC68376, SKF-86002, a dominant-negative mutant of p38α, and a dominant-negative mutant of p38 β.

5. The method of claim 1 wherein the inhibitor of p38 MAP kinase is an antisense oligonucleotide.

6. The method of claim 1 wherein the inhibitor of p38 MAP kinase is a vector which expresses a protein or polypeptide which inhibits p38 MAP kinase.

7. The method of claim 1 wherein the method of administration is selected from the group consisting of: inhalation, ear drops, transtympanically, intramuscularly, intravenously, and by mouth.

8. A method for the identification of regulators of mucin production, comprising:
providing a reporter vector containing the MUC5AC or p38 MAP kinase promoter;
contacting the reporter vector with a potential regulator; and
identifying the up-or down-regulation of the reporter gene.

9. The method of claim 8, wherein said potential regulator is selected from the group consisting of: a polypeptide, an polynucleotide, and a small molecule.

10. The method of claim 8, wherein said potential regulator is a mixture of proteins from a cell.

11. The method of claim 8, wherein said potential regulator is an antisense polynucleotide.

12. The method of claim 8, wherein said potential regulator is a library of small molecules.

13. A method for the treatment of overproduction of mucin in a mammal, comprising:
administering an activator of PI-3 kinase to the mammal in an amount sufficient to reduce mucin production.

14. The method of claim 13 wherein the overproduction of mucin is caused by a disease selected from the group consisting of: Otitis media, chronic obstructive pulmonary disease, asthma, and cystic fibrosis.

15. The method of claim 14, wherein said overproduction of mucin is caused by otitis media (OM) infection or chronic obstructive pulmonary disease (COPD).

16. The method of claim 14 wherein the OM or COPD is cause by nontypeable Haemophilus influenzae (NTHi).

17. The method of claim 13 wherein said activator of PI-3 kinase is a protein selected from the group consisting of: a dominant negative mutant of PI-3 kinase, a constitutively active form of p110 (p110-CAAX), wildtype Akt.

18. The method of claim 13 wherein the inhibitor of p38 MAP kinase is an antisense oligonucleotide.

19. The method of claim 13 wherein the inhibitor of PI-3 kinase is a vector which expresses a protein or polypeptide which activates PI-3 kinase.

20. The method of claim 13 wherein the method of administration is selected from the group consisting of: inhalation, ear drops transtympanically, intramuscularly, intravenously, and by mouth.
